(19) United States
(12) Patent Application Publication
(10) Pub. No.: US 2022/0126046 A1
(43) Pub. Date: Apr. 28, 2022

(54) Rechargeable Hoseless CPAP System

(71) Applicant: John D. King, Knoxville, TN (US)

(72) Inventor: John D. King, Knoxville, TN (US)

(21) Appl. No.: 17/509,420

(22) Filed: Oct. 25, 2021

Related U.S. Application Data
(60) Provisional application No. 63/104,831, filed on Oct. 23, 2020.

(51) Int. Cl. A61M 16/06; A61M 16/10; A61M 16/00

(52) U.S. Cl. CPC A61M 16/0605; A61M 2205/8206; A61M 16/0003; A61M 16/1055

(57) ABSTRACT

A hoseless CPAP system having a hoseless CPAP mask including a housing, a sealing portion extending from a back of the housing and configured to fit over a nose and mouth of a user, a fan unit provided in the housing to force air through the sealing portion to be breathed by the user, a battery provided in the housing and configured to power the fan unit, and one or more electrical contacts provided on the housing to provide charging power to the battery; and a charging base including a receiving portion configured to hold the CPAP mask therein, and one or more electrical contacts provided in the receiving portion and configured to contact the one or more electrical contacts on the CPAP mask housing to provide charging power to the battery.

### CROSS-REFERENCE TO RELATED APPLICATIONS

This application claims the benefit of U.S. Provisional Patent Application Ser. No. 63/104,831, filed on Oct. 23, 2020, which is incorporated herein in its entirety by reference.

### FIELD OF INVENTION

The present general inventive concept relates to a continuous positive airway pressure (CPAP) system, and, more particularly, to an easily portable, rechargeable, and AC/DC hoseless CPAP system.

### BACKGROUND

As several millions of Americans have been diagnosed with obstructive sleep apnea (OSA), and with more and more being diagnosed every year, continuous positive airway pressure (CPAP) machines are becoming more and more commonplace in people's homes and lives. There were approximately 8.1 million users in the United States in one recent year, and it is believed that about 4-6 million others are in need but go undiagnosed. OSA causes sufferers to have repeated and intermittent interruptions in breathing, due to the airway at the back of the throat becoming blocked during sleep. This has detrimental long-term health effects, aside from the simple lack of quality sleep that occurs at night. The conventional CPAP machine works by delivering pressurized air through a hose and mask into the wearer's airway during sleep. Due to the steady flow of the pressurized air, the airway is kept open, thereby improving respiration and sleep quality.

However, while these respiratory benefits are important in helping a wearer achieve better sleep through better breathing, the conventional CPAP machine is a terribly unwieldy and obtrusive system. Such a CPAP machine, an example of which is illustrated in FIG. 1, includes a rather large base unit 100 that requires a stable and flat surface to rest upon, has an AC power source 110 which must be plugged into a wall socket, and a large hose 120 that connects the base unit 100 to a mask worn 130 by the user. Such a configuration results in a significant physical intrusion to the user's comfort in bed, at least due to the hose 120 being very restrictive to the positions and movement of the wearer. Further, the number of components and general bulkiness of the conventional CPAP system makes it incredibly inconvenient to carry with the user to operate at remote locations during travel.

Therefore, it would be desirable to provide a CPAP system that allows a user to enjoy the benefit of improved breathing during sleep without the constraints of being tethered by a large hose to a base unit, and of being conveniently portable.

### BRIEF SUMMARY

According to various example embodiments of the present general inventive concept, a hoseless CPAP system is provided to allow a user to comfortably and conveniently wear a battery powered CPAP mask with no external hose forcing air therethrough, and having a charging base in which to house the hoseless CPAP mask and charge the mask battery. In some embodiments CPAP mask use and/or operational data may be accessed through electrical contacts provided in the charging base.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by providing a hoseless CPAP system including a hoseless CPAP mask having a housing, a sealing portion extending from a back of the housing and configured to fit over a nose and mouth of a user, a fan unit provided in the housing to force air through the sealing portion to be breathed by the user, a battery provided in the housing and configured to power the fan unit, and one or more electrical contacts provided on the housing to provide charging power to the battery; and a charging base having a receiving portion configured to hold the CPAP mask therein, and one or more electrical contacts provided in the receiving portion and configured to contact the one or more electrical contacts on the CPAP mask housing to provide charging power to the battery.

The foregoing and/or other aspects and advantages of the present general inventive concept may also be achieved by providing a hoseless CPAP mask including a housing, a sealing portion extending from a back of the housing and configured to fit over a nose and mouth of a user, a fan unit provided in the housing to force air through the sealing portion to be breathed by the user, a battery provided in the housing and configured to power the fan unit, and one or more electrical contacts provided on the housing to provide charging power to the battery

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a conventional CPAP machine;

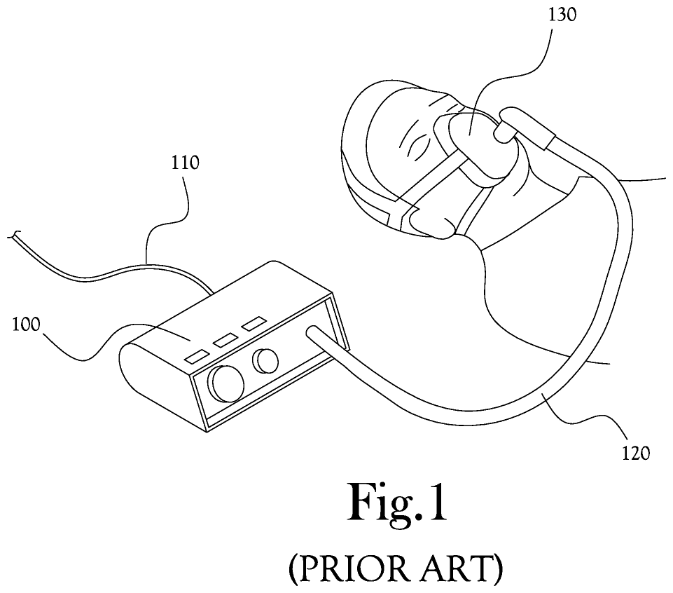

FIG. 2 illustrates a hoseless CPAP system according to an example embodiment of the present general inventive concept;

FIG. 3 illustrates a perspective view of the mask of the hoseless CPAP system of FIG. 2, with phantom lines illustrating the inner componentry, according to an example embodiment of the present general inventive concept;

FIGS. 4A-D respectively illustrate front, back, top, and bottom views of the mask of FIG. 3;

FIGS. 5A-B illustrate the placement of the mask of FIG. 3 on the head of a user according to an example embodiment of the present general inventive concept;

FIGS. 6A-B illustrate various views of the charger of the hoseless CPAP system of FIG. 2 according to an example embodiment of the present general inventive concept;

FIG. 7 illustrates a schematic view of various components of the mask of FIG. 3 according to an example embodiment of the present general inventive concept; and

FIG. 8 illustrates a schematic view of various components of the charger of FIGS. 6A-B according to an example embodiment of the present general inventive concept.

### DETAILED DESCRIPTION

Reference will now be made to the example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order.

Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features.

Thus, the exemplary term “below” can encompass both an orientation of above and below. The device may be otherwise oriented (rotated 90 degrees or at other orientations) and the spatially relative descriptors used herein interpreted accordingly.

According to various example embodiments of the present general inventive concept, a hoseless CPAP system is provided that increases the convenience of application and operation for the user, and that is readily portable so that the user can conveniently carry and use the system during travel. In various example embodiments the CPAP system includes a CPAP mask that does not require a hose or air circulating base connected thereto, but rather includes a fan unit in the mask to provide the forced air to the user. Various example embodiments may also include a charger that serves as a docking station for the mask when not in use. Various example embodiments are provided with a rechargeable power pack that allows a user to enjoy the benefit of improved breathing during sleep, for example, for 3-5 nights without charging when no traditional power source is available, and without the constraints of being tethered by a large hose to a base unit, all while being conveniently portable.

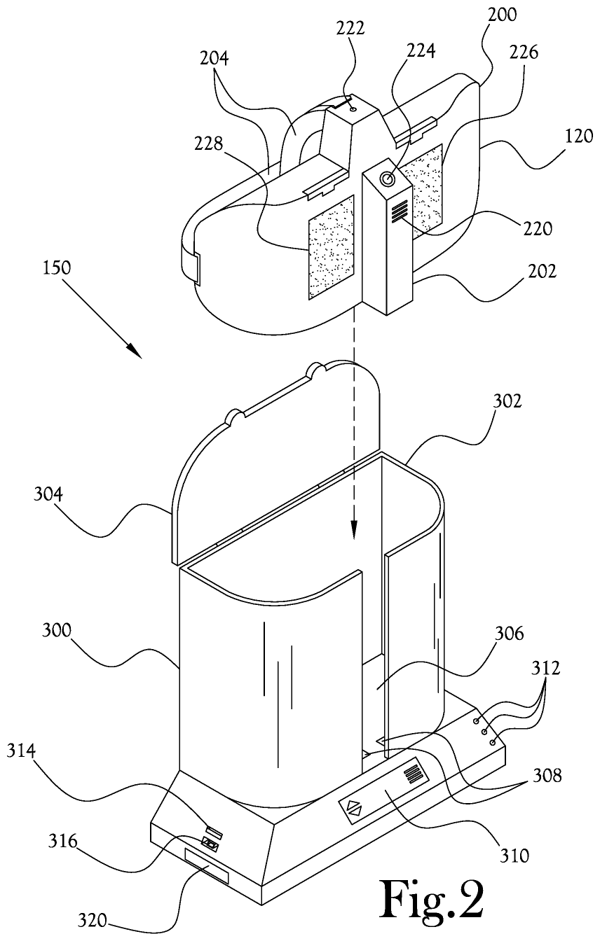

FIG. 2 illustrates a hoseless CPAP system according to an example embodiment of the present general inventive concept. As illustrated in FIG. 2, the CPAP system 150 according to this example embodiment of the present general inventive concept includes a hoseless CPAP mask 200, which may be referred to generally herein simply as the mask 200, configured to fit into a charging station 300 that also operates as a docking station and data transfer base, referred to generally herein as the charger 300. The charger 300 has an enclosure 302 with a lid 304 that is able to enclose the mask 200 and sanitize the mask 200 with, for example, UV light (UV-A, UV-B, UV-C, and so on) and/or other sanitizers, during a charging operation. In various example embodiments, a UV-C LED is provided in the enclosure 302 to provide UV-C light for sanitizing the mask 200. In various example embodiments a UV-C LED may be provided in the overall housing of the mask 200. The enclosure 302 protects the mask 200 from dust or other contaminants during charging and/or storage. In the example embodiment illustrated in FIG. 2, a gap 306 is formed in the enclosure 302 to allow the protruding front portion 202 of the mask 200 to fit therein. Various other example embodiments may have various different configurations of the mask and/or charger encasement without departing from the scope of the present general inventive concept. The electrical contacts 308 are arranged inside the enclosure 302 to connect with corresponding contacts on the bottom of the mask 200 to allow charging of the mask's battery, as well as facilitating data transfer about the user's sleep from the mask 200 to the charger 300 in some example embodiments. The gap 306 of this example embodiment is formed so as to correspond to the protruding front portion 202 of the mask 200 such that the front portion 202 extends therethrough and can function as a de facto handle to lower the mask 200 into charger 300, as well as to lift the mask 200 out of the charger 300. The fitting of the front portion 202 in the gap 306 also aids in the alignment of the electrical contacts of the charger 300 and the mask 200. Such a configuration and fitting may also conserve space and allow the interaction of the protruding front portion 202 and the gap 306 to stabilize the mask 200 inside the charger 300.

As illustrated in FIG. 2, the charger 300 includes an interactive display 310 to allow a user to choose various operations and see information assorted therewith. A plurality of indicators 312 may also be provided on the face of the charger to indicate various operations/states such as, for example, “CHARGED”, “CHARGING”, and “UV SANITIZER”. A USB port 314 is provided on the charger 300 to allow data transfer to and from the mask 200 from another device such as a computer, cell phone, etc., and which may also be used as a charger for other USB connected devices such as a cell phone. The charger 300 includes a switch 316 that can be switched between power and data operations for the USB port 314. Sleep data associated with the user, and recorded by the mask 200, can be downloaded to, for example, a zip drive through the USB port 314. In various example embodiments the charger 300 may include a transmitter to send such data through a wireless connection, such as, for example, Bluetooth, to the user's sleep physician. The sanitizing feature of the system 150 utilizes LED UV-C light located in both the charger 300 and the mask 200 in various example embodiments, so that the system 150 may be cleaned while not in use and while in operation, the air being cleaned/sanitized in real time during use of the mask.

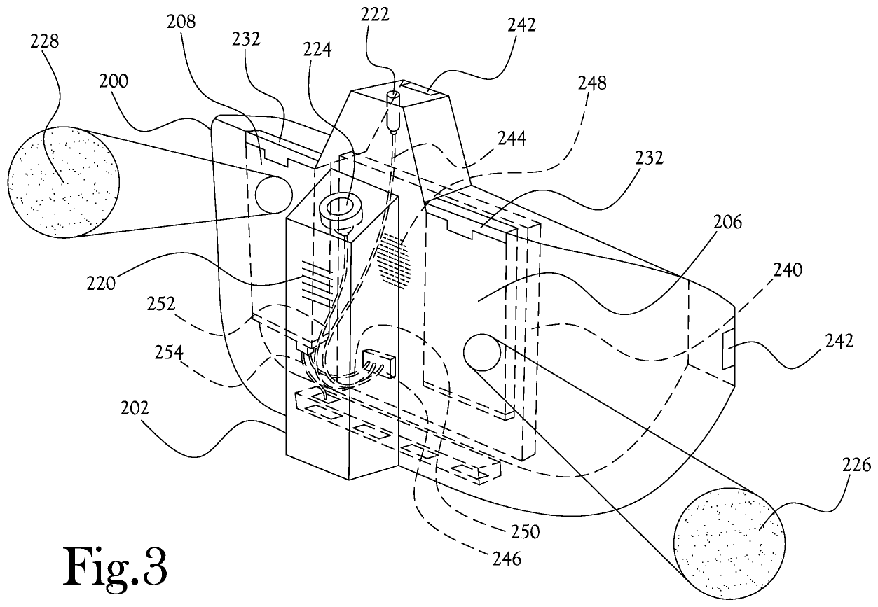

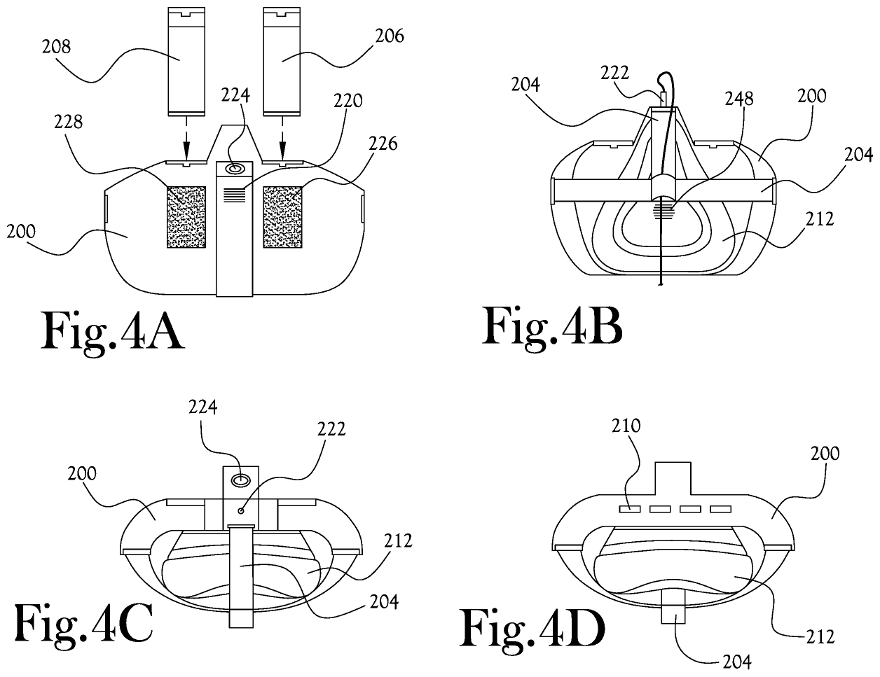

As illustrated in FIG. 2, the mask 200 is selectively removable from the charger 300 to be worn by the user during sleep. Along with the perspective view of the mask 200 illustrated in FIG. 2, FIG. 3 illustrates a perspective view of the mask 200 of FIG. 2 with phantom lines illustrating the inner componentry, according to an example embodiment of the present general inventive concept, and FIGS. 4A-D respectively illustrate front, back, top, and bottom views of the mask 200. The mask 200 includes retractable straps 204 that may have a pull tab to allow easy fitting of the mask 200 on the head of the user. The mask 200 includes a removable and washable air filter 206 for incoming air, and an HME (Humidity Moisture Exchange) filter, to filter the forced air before being expelled through the exhaust 220. The washable air filter 206 may be provided adjacent to an air intake port 226, formed here as perforations, provided on a front of the mask 200. A fan 240 in the mask draws air in through the air intake port 226 and through the air filter 206 to be delivered to the user through the mouth/nosepiece. The HME filter may be arranged in the protruding front portion 202 of the mask 200 proximate the exhaust 220, and captures heat and moisture as the user exhales and returns it back during breathing, thus eliminating the need for an external HME filter such as used in conventional systems. The protruding front portion 202, or “beak” of the mask, may be formed to be removable, such as with a sliding connection, for removal and to create access to the inner chamber holding the HME filter (not illustrated, for sake of clarity of other componentry in FIG. 3). Various example embodiments may include hygienic and cleanable synthetic foam mesh pads in the mask as an HME filter, while other example embodiments may include a disposable cartridge or similar material as the HME filter. Such a disposable cartridge may have a life span of 30-40 days. The HME filter eliminates the need for a tank as in the conventional system. The HME filter is in line with the forced air in the mask 200, and is able to capture humidity to keep moisture in the system and prevent the drying of the user's breath pathway. In various example embodiments the filter can be used for as long as two weeks without needing to be washed. The mask 200 includes a rechargeable lithium ion battery 208 that may be charged when the mask 200 is stored in the charger 300 and a plurality of electrical contacts 210 of the mask 200 are in contact with the corresponding electrical contacts 308 of the charger 300. As illustrated in FIG. 3, the battery 208 may be arranged so as to be adjacent to a perforated port 228 that allows the escape of heat that may build up from the battery 208 during operation. The mask 200 may be equipped with a low battery alarm to indicate, through visual and/or audible cues, that the battery has been depleted to a predetermined level. As illustrated in FIG. 4B, the sealing portion 212 of the mask 200 fits over the nose and mouth of the user to allow air forced by the fan 240 provided in the mask 200 to force air through the air opening 248 to the user's breath pathway. In various example embodiments the mask 200 may be constructed of a lightweight material such as an ABS plastic or polycarbonate plastic, making the mask 200 comfortable to wear after installing the compressor/pump, air filter, battery, retractable straps, etc. The retractable straps 204 may be anchored to the mask by strap connections 242 as shown in FIGS. 3 and 4B-C, and may be designed to be removable for servicing/replacing, and may be adjustable to accommodate different levels of fit for different users. In various example embodiments the mask 200 may include an optional AC adaptor for use with a different power source. In the example embodiment illustrated in FIG. 4B, an auxiliary power jack 222 is formed on a top portion of the mask to receive optional power through a connecting line with the charger 300, or from an adapter plugged into a wall outlet, etc. The mask 200 has an on/off button 224 that may be integrated with the beak 202 of the mask 200, or may in various example embodiments may be formed on different portions of the mask 200. FIG. 3 illustrates a power wire 244 that provides electrical communication between the auxiliary power jack 222 and an electronic control unit (ECU) 246 of the electric fan 240, a power wire 250 that provides electrical communication between the battery 208 and the ECU 246, a wire 252 that provides electrical communication between the on/off button 224 and the ECU 246, and a wire 254 that provides electrical communication between the electrical contacts 210 and the rechargeable battery 208. The mask 200 may be programmable for a single pressure of air, for variable pressure, an optimal ramp-up time, and so on.

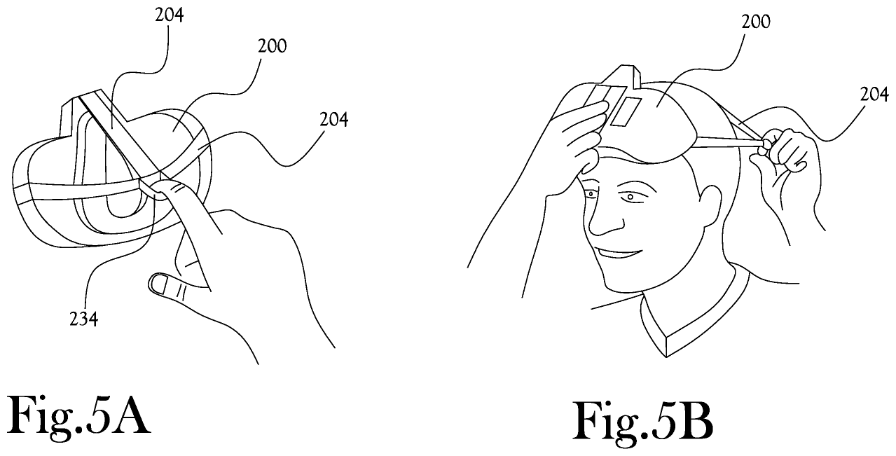

As illustrated in FIG. 4A, the mask 200 may be configured such that the air filter 206 and battery 208 are removable, in a modular fashion, from the mask 200 by being inserted and removed from corresponding slots configured to hold the air filter 206 and battery 208 securely therein. In various example embodiments selectively openable tabs or lids 232 may be provided at the tops of those slots to hold the respective air filter 206 and battery 208 therein. FIGS. 5A-B illustrate the placement of the mask of FIG. 3 on the head of a user according to an example embodiment of the present general inventive concept. As illustrated, elastic straps 204 may be provided with a tab 234 or other such easily gripped portion that a user can grab, place a finger inside, etc., to stretch the straps 204 over and around the user's head. The user can grip the protruding front portion 202 with the other hand to conveniently place the mask 200 over the nose and mouth, where it is held in place by the straps 204 extending around and over the user's head.

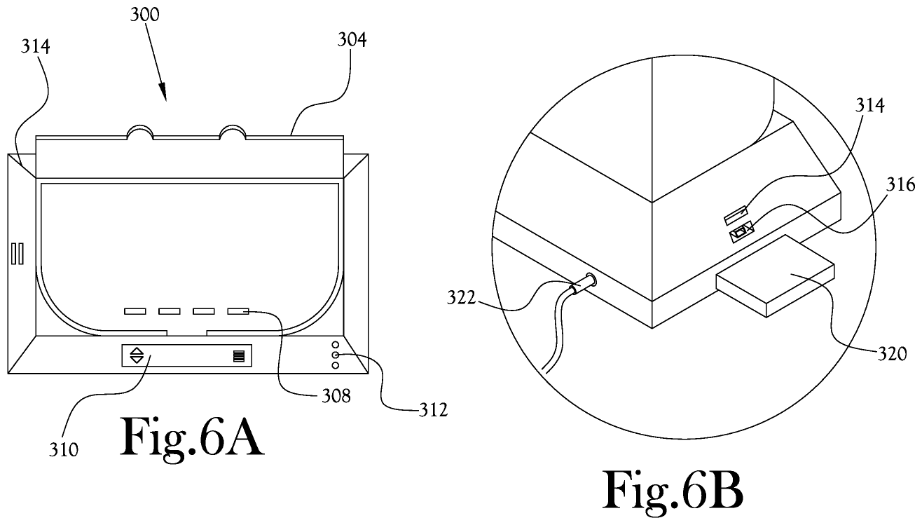

FIGS. 6A-B illustrate various views of the charger of the hoseless CPAP system of FIG. 2 according to an example embodiment of the present general inventive concept. Along with the features already discussed in regard to FIG. 2, the charger 300 may also include an AC power jack 322 to provide AC power to the charger 300, and a removable battery 320 to power the charger when AC power may not be available. The display/UI 310 may be an LED screen. The charger 300 may provide automatic start and turn off for sanitizing and charging operations.

The battery 320 may be a 50K mAh power pack which may allow the recharging of the mask 22 6-8 times during power outages, camping, etc., when AC power is not available to the charger 300. The receiving portion for the battery 320 may be of the spring-loaded type which allows the user to push the battery 320 to click into place, and to have the battery 320 ejected. The lid 304 may be hinged so as to be easily opened and closed to place the mask 200 inside the charger 300, or to remove the mask 200 from the charger 300, and may contain most of the sanitizing UV light therein.

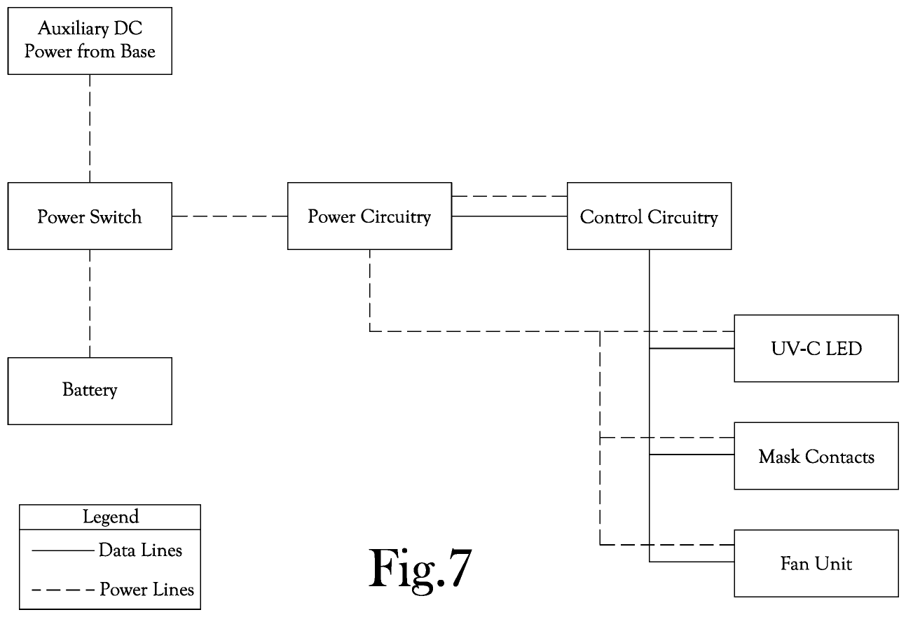

FIG. 7 illustrates a schematic view of various components of the mask of FIG. 3 according to an example embodiment of the present general inventive concept. As illustrated in FIG. 7, the mask 200 includes power and control circuitry which drive and/or control the UV-C LED in the mask 200, the electrical contacts 210 of the mask 200, and the fan unit provided in the mask 200. Data lines are shown as solid lines in FIG. 7, and power lines are shown as broken lines. As illustrated, the power circuitry is connected to a power switch located on the mask 200, which allows the use, and charging of, the battery. Additionally, auxiliary DC power may be drawn from the base, through the USB connections, to power the mask 200 if the battery is not able to do so. The control circuitry may comprise all necessary passive devices, MCU's, and FPGA's to effectively control the onboard components. The power circuitry may contain all necessary passive devices, PMIC's, and regulators to provide required voltages and current for the onboard components.

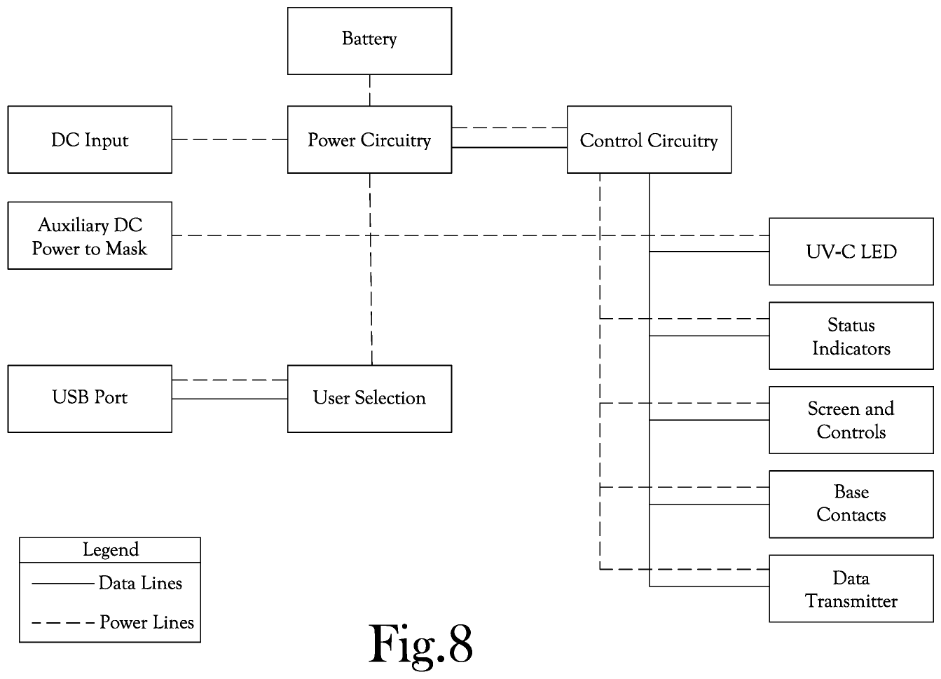

FIG. 8 illustrates a schematic view of various components of the charger of FIGS. 6A-B according to an example embodiment of the present general inventive concept. As illustrated in FIG. 8, the charger 300 or base includes power and control circuitry which drive and/or control the UV-C LED in the charger 300, the status indicators 312, the screen and UI controls 310, the electrical contacts 308 of the charger 300, and a data transmitter. Power may be provided to the power circuitry and other various components by the DC input, and may also pass the power through to an auxiliary power jack provided on the mask in the event that the onboard battery of the mask is not sufficiently charged. The user selection serves to provide control input to the screen/UI controls 310 and the USB port 314. Data lines are shown as solid lines in the FIG. 6, and power lines are shown as broken lines. The control circuitry may comprise all necessary passive devices, MCU's, and FPGA's to effectively control the onboard components. The power circuitry may contain all necessary passive devices, PMIC's, and regulators to provide required voltages and current for the onboard components. The control circuitry in the charger 300 and the mask 200 that may include the necessary memory for any programming or storage function being initiated at the charger 300 through the user interface screen 310. Sending any necessary updates, changes, etc., to the control circuitry in the mask can be transmitted from the control circuitry in the charger via the data/electrical contacts provided to the charger and mask. This data path may be reversed when sending sleep data from the mask back to the charger for memory storage. Such information may be sent in some example embodiments via WiFi of Bluetooth via the transmitter located in the base unit to the user's sleep doctor monthly. In various example embodiments the same data can also be downloaded onto a thumb drive via the USB port located on the charger.

Various example embodiments of the present general inventive concept may provide a hoseless CPAP system including a mask configured to provide a sealed fit over a user's nose and mouth, a fan unit provided in the mask to force air through the mask to be breathed by the user, and a charging base configured to electrically charge a battery in the mask when the mask is in electrical contact with the charging base. The charging base may include an enclosure configured to at least partially enclose the mask when the mask is in electrical contact with the charging base, the enclosure include a UV light source to sanitize the mask. The charging base may include a USB port configured to selectively provide power to a connected component and to provide data transfer from the mask to a connected component. The system may further include an HME filter proximate an exhaust of the mask to capture heat and moisture form exhaled breath and return it to an air intake port of the mask. The system may further include a UV light source on the mask to provide sanitizing of the mask during use of the mask.

Various example embodiments of the present general inventive concept may provide a hoseless CPAP system including a hoseless CPAP mask having a housing, a sealing portion extending from a back of the housing and configured to fit over a nose and mouth of a user, a fan unit provided in the housing to force air through the sealing portion to be breathed by the user, a battery provided in the housing and configured to power the fan unit, and one or more electrical contacts provided on the housing to provide charging power to the battery; and a charging base having a receiving portion configured to hold the CPAP mask therein, and one or more electrical contacts provided in the receiving portion and configured to contact the one or more electrical contacts on the CPAP mask housing to provide charging power to the battery. The CPAP mask may further include an air intake portion formed on the housing to pass air from outside the housing to the fan unit, and an air filter provided between the air intake portion and the fan unit, wherein the air filter is configured to be selectively removable from the housing. The CPAP mask may further include one or more straps coupled to the housing and configured to provide a secure fit to a head of the user, wherein the one or more straps are formed with elastic properties. The CPAP mask may further include an air output portion formed on the housing to pass exhaled air from the sealing portion outside the housing. The system may further include a filter provided in the housing between the sealing portion and the air output portion. The CPAP mask may further include one or more vent openings formed in the housing proximate the battery to dissipate heat to outside the housing. The charging base may further include a UV-light source to sanitize the CPAP mask when the CPAP mask is received in the charging base. The charging base may further include a USB port in electrical communication with the electrical contacts of the charging base to send and receive data signals between the CPAP mask and an external processing device. The charging base may include at least a partial enclosure to receive the CPAP mask therein. The partial enclosure may be formed with a slot in a front portion thereof, and the housing of the CPAP mask may be formed with a protruding front portion that is configured to slide through the slot of the partial enclosure and extend out of the partial enclosure when the CPAP mask is received therein. The charging base may be configured to receive AC power to operate the charging base and to charge the battery of the CPAP mask. The charging base may further include a removable battery to provide power to operate the charging base and to charge the battery of the CPAP mask in lieu of AC power. The charging base may further include an electronic display to indicate various operating states of the system. The CPAP mask may further include an auxiliary power jack to receive AC power to operate the CPAP mask in lieu of battery power.

Various example embodiments of the present general inventive concept may provide a hoseless CPAP mask including a housing, a sealing portion extending from a back of the housing and configured to fit over a nose and mouth of a user, a fan unit provided in the housing to force air through the sealing portion to be breathed by the user, a battery provided in the housing and configured to power the fan unit, and one or more electrical contacts provided on the housing to provide charging power to the battery. The hoseless CPAP mask may further include an air intake portion formed on the housing to pass air from outside the housing to the fan unit, and an air filter provided between the air intake portion and the fan unit, wherein the air filter is configured to be selectively removable from the housing. The hoseless CPAP mask may further include one or more straps coupled to the housing and configured to provide a secure fit to a head of the user, wherein the one or more straps are formed with elastic properties. The hoseless CPAP mask may further include an air output portion formed on the housing to pass exhaled air from the sealing portion outside the housing. The hoseless CPAP mask may further include a filter provided in the housing between the sealing portion and the air output portion. The hoseless CPAP mask may further include one or more vent openings formed in the housing proximate the battery to dissipate heat to outside the housing.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein, using sound engineering judgment.

Numerous variations, modification, and additional embodiments are possible, and, accordingly, all such variations, modifications, and embodiments are to be regarded as being within the spirit and scope of the present general inventive concept.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

1. A hoseless CPAP system, comprising:
a hoseless CPAP mask comprising:
a housing,
a sealing portion extending from a back of the housing and configured to fit over a nose and mouth of a user,
a fan unit provided in the housing to force air through the sealing portion to be breathed by the user,
a battery provided in the housing and configured to power the fan unit, and
one or more electrical contacts provided on the housing to provide charging power to the battery; and
a charging base comprising:
a receiving portion configured to hold the CPAP mask therein, and
one or more electrical contacts provided in the receiving portion and configured to contact the one or more electrical contacts on the CPAP mask housing to provide charging power to the battery.

2. The system of claim 1, wherein the CPAP mask further comprises:
an air intake portion formed on the housing to pass air from outside the housing to the fan unit; and
an air filter provided between the air intake portion and the fan unit;
wherein the air filter is configured to be selectively removable from the housing.

3. The system of claim 1, wherein the CPAP mask further comprises:
one or more straps coupled to the housing and configured to provide a secure fit to a head of the user;
wherein the one or more straps are formed with elastic properties.

4. The system of claim 1, wherein the CPAP mask further comprises:
an air output portion formed on the housing to pass exhaled air from the sealing portion outside the housing.

5. The system of claim 4, further comprising a filter provided in the housing between the sealing portion and the air output portion.

6. The system of claim 1, wherein the CPAP mask further comprises one or more vent openings formed in the housing proximate the battery to dissipate heat to outside the housing.

7. The system of claim 1, wherein the charging base further comprises a UV-light source to sanitize the CPAP mask when the CPAP mask is received in the charging base.

8. The system of claim 1, wherein the charging base further comprises a USB port in electrical communication with the electrical contacts of the charging base to send and receive data signals between the CPAP mask and an external processing device.

9. The system of claim 1, wherein the charging base comprises at least a partial enclosure to receive the CPAP mask therein.

10. The system of claim 9, wherein the partial enclosure is formed with a slot in a front portion thereof; and
wherein the housing of the CPAP mask is formed with a protruding front portion that is configured to slide through the slot of the partial enclosure and extend out of the partial enclosure when the CPAP mask is received therein.

11. The system of claim 1, wherein the charging base is configured to receive AC power to operate the charging base and to charge the battery of the CPAP mask.

12. The system of claim 11, wherein the charging base further comprises a removable battery to provide power to operate the charging base and to charge the battery of the CPAP mask in lieu of AC power.

13. The system of claim 1, wherein the charging base further comprises an electronic display to indicate various operating states of the system.

14. The system of claim 1, wherein the CPAP mask further comprises an auxiliary power jack to receive AC power to operate the CPAP mask in lieu of battery power.

15. A hoseless CPAP mask, comprising:
a housing;
a sealing portion extending from a back of the housing and configured to fit over a nose and mouth of a user;
a fan unit provided in the housing to force air through the sealing portion to be breathed by the user;
a battery provided in the housing and configured to power the fan unit; and
one or more electrical contacts provided on the housing to provide charging power to the battery.

16. The hoseless CPAP mask of claim 15, further comprising:
an air intake portion formed on the housing to pass air from outside the housing to the fan unit; and
an air filter provided between the air intake portion and the fan unit;
wherein the air filter is configured to be selectively removable from the housing.

17. The hoseless CPAP mask of claim 15, further comprising:
one or more straps coupled to the housing and configured to provide a secure fit to a head of the user;
wherein the one or more straps are formed with elastic properties.

18. The hoseless CPAP mask of claim 15, further comprising:
an air output portion formed on the housing to pass exhaled air from the sealing portion outside the housing.

19. The hoseless CPAP mask of claim 18, further comprising a filter provided in the housing between the sealing portion and the air output portion.

20. The hoseless CPAP mask of claim 15, further comprising one or more vent openings formed in the housing proximate the battery to dissipate heat to outside the housing.
